(19) United States
(12) Patent Application Publication
(10) Pub. No.: US 2025/0040896 A1
(43) Pub. Date: Feb. 6, 2025

(54) MEDICAL IMAGING METHOD AND APPARATUS AND MEDICAL IMAGING DEVICE

(71) Applicant: GE Precision Healthcare LLC, Waukesha, WI (US)

(72) Inventors: Xueli Wang, Beijing (CN); Bingjie Zhao, Beijing (CN); Yuwen Dong, Beijing (CN)

(21) Appl. No.: 18/788,872

(22) Filed: Jul. 30, 2024

(30) Foreign Application Priority Data
Jul. 31, 2023 (CN) 202310953306.8

(51) Int. Cl. A61B 6/04; A61B 6/00; A61B 6/46; G06T 7/13; G06T 7/60

(52) U.S. Cl. CPC A61B 6/0487; A61B 6/0407; A61B 6/0492; A61B 6/461; A61B 6/488; G06T 7/13; G06T 7/60; G06T 2207/10081; G06T 2207/10116; G06T 2207/20084; G06T 2207/30004

(57) ABSTRACT

Embodiments of the present application provide a medical imaging method and apparatus. The method includes acquiring a region of interest on a scout image of a subject using a deep learning method, determining a geometric center of an organ of interest of the subject according to the region of interest, and, according to the geometric center of the organ of interest and a geometric rotation center of a medical imaging device, driving a table to move. According to the embodiments of the present application, automatic movement of the table is achieved so that the geometric center of the organ of interest is close to the geometric rotation center of the medical imaging device, enabling diagnostic images to have higher image quality, including a higher image resolution. In addition, rays passing through the geometric rotation center of the medical imaging device are fully utilized, improving the dose efficiency of the rays.

### CROSS-REFERENCE TO RELATED APPLICATIONS

This application claims priority to Chinese Application No. 202310953306.8, filed on Jul. 31, 2023, the disclosure of which is incorporated herein by reference in its entirety.

### TECHNICAL FIELD

Embodiments of the present application relate to the technical field of medical devices, and in particular to a medical imaging method and apparatus and a medical imaging device.

### BACKGROUND

Computed tomography (CT) utilizes accurately collimated X-ray beams or gamma rays, together with a highly sensitive detector, to perform cross-sectional scans one by one around a certain site of the human body; it is characterized by short scanning time, image clarity, and the like, and can be used to detect a wide range of diseases. Depending on the different rays used, CT can be classified as: X-ray CT (X-CT), gamma-ray CT (gamma-CT), and so on.

CT tomography has many advantages. First, CT tomography can obtain images having high density and resolution, so that doctors can clearly observe information such as the size, shape and position of a lesion site. Second, CT scanning is non-invasive and has a relatively low radiation dose, thereby offering a high level of safety for patients. In addition, CT scanning has high speed and is suitable for scenarios requiring rapid diagnosis, such as emergency treatment.

CT tomography has a wide range of clinical applications. In the diagnosis of head and neck diseases, CT scanning can help doctors observe skull and soft tissue structures to locate diseased tissues. In the diagnosis of thoracic diseases, CT scanning can be used to observe lung and heart structures, so as to detect diseases such as lung cancer, pneumonia and pneumothorax. In the diagnosis of abdominal diseases, CT scanning can be used to observe the structures of organs such as the liver, spleen and kidney, so as to detect diseases such as tumors and calculi. In addition, CT tomography may also play a role in the diagnosis of bone and spinal diseases.

It should be noted that the above introduction of the background is only for the convenience of clearly and completely describing the technical solutions of the present application, and for the convenience of understanding for those skilled in the art.

### SUMMARY

The inventors have found that, in a CT scanning process, a patient (subject under detection) first needs to be placed on a patient table (also referred to as a scanning table, a CT table, or the like), and then the patient table is manually operated, for example, moved back and forth or moved up and down, and the positioning is implemented with the aid of a laser light. However, the site to be examined, such as the heart or spine, is located within the body of the patient, and the position of the site varies from patient to patient and is not at the geometric center of the contour of the patient. As a result, regardless of whether the patient table is moved back and forth or up and down, it is impossible to move the site to be examined (i.e., an organ of interest) to the geometric rotation center of a medical imaging device (i.e., a CT gantry). That is, manually operating the patient table results in low operation efficiency and low movement accuracy.

In view of at least one of the above technical problems or other similar problems, the embodiments of the present application provide a medical imaging method and apparatus, and a medical imaging device, used to achieve automatic movement of a patient table, thereby improving operation efficiency and movement accuracy.

According to one aspect of the embodiments of the present application, a medical imaging method is provided. The method includes acquiring a region of interest on a scout image of an examination subject using a deep learning method, determining a geometric center of an organ of interest of the examination subject according to the region of interest, and according to the geometric center of the organ of interest and a geometric rotation center of a medical imaging device, driving a patient table to move.

According to another aspect of the embodiments of the present application, a medical imaging apparatus is provided. The apparatus includes an acquisition unit, which acquires a region of interest on a scout image of an examination subject using a deep learning method, a first determination unit, which determines a geometric center of an organ of interest of the examination subject according to the region of interest, and a control unit, which, according to the geometric center of the organ of interest and a geometric rotation center of a medical imaging device, drives a patient table to move.

According to another aspect of the embodiments of the present application, a medical imaging device is provided. The medical imaging device comprises a memory and a processor. The memory stores a computer program, and the processor is configured to execute the computer program to implement the medical imaging method described above.

One of the beneficial effects of the embodiments of the present application is that, according to the embodiments of the present application, automatic movement of the patient table is achieved, so that the geometric center of the organ of interest is close to the geometric rotation center of the medical imaging device, enabling diagnostic images to have higher image quality, including a higher image resolution. In addition, rays passing through the geometric rotation center of the medical imaging device are fully utilized, accordingly improving the dose efficiency of the rays.

With reference to the following description and drawings, specific embodiments of the embodiments of the present application are disclosed in detail, and the means by which the principles of the embodiments of the present application can be employed are illustrated. It should be understood that the embodiments of the present application are therefore not limited in scope. Within the scope of the spirit and clauses of the appended claims, the embodiments of the present application comprise many changes, modifications, and equivalents.

### BRIEF DESCRIPTION OF THE DRAWINGS

The included drawings are used to provide further understanding of the embodiments of the present application, which constitute a part of the description and are used to illustrate the embodiments of the present application and explain the principles of the present application together with textual description. Evidently, the drawings in the following description are merely some embodiments of the present application, and those of ordinary skill in the art may obtain other embodiments according to the drawings without involving inventive skill. In the drawings:

FIG. 1 is a schematic diagram of a CT imaging device according to an embodiment of the present application;

FIG. 2 is a schematic diagram of a CT imaging system according to an embodiment of the present application;

FIG. 3 is a schematic diagram of a medical imaging method according to an embodiment of the present application;

FIG. 4 is a schematic diagram of a scout image of the chest;

FIG. 5 is another schematic diagram of a scout image of the chest;

FIG. 6 is a schematic diagram of a bounding box B1 for an organ of interest, said bounding box being obtained by performing size transformation and normalization on an input scout image, and then performing feature extraction on the scout image by means of a deep learning network;

FIG. 7 is a schematic diagram of a bounding box B2 for an organ of interest, said bounding box being obtained by performing feature extraction on an input scout image by means of a deep learning network to obtain a contour C1 of the organ of interest, and then converting the contour C1;

FIG. 8 is a schematic diagram of a bounding box for an organ of interest obtained based on the 0-degree scout image shown in FIG. 4;

FIG. 9 is a schematic diagram of a bounding box for an organ of interest obtained based on the 90-degree scout image shown in FIG. 5;

FIG. 10 is a schematic diagram of a center line obtained by intersecting a tangent plane of a center line of a first bounding box with a tangent plane of a center line of a second bounding box;

FIG. 11 is a schematic diagram of a display field-of-view adjustment strategy;

FIG. 12 is a schematic diagram of a medical imaging apparatus according to an embodiment of the present application; and

FIG. 13 is a schematic diagram of a medical imaging device according to an embodiment of the present application.

### DETAILED DESCRIPTION

The foregoing and other features of the embodiments of the present application will become apparent from the following description and with reference to the drawings. In the description and drawings, specific embodiments of the present application are disclosed in detail, and part of the embodiments in which the principles of the embodiments of the present application may be employed are indicated. It should be understood that the present application is not limited to the described embodiments. On the contrary, the embodiments of the present application include all modifications, variations, and equivalents which fall within the scope of the appended claims.

In the embodiments of the present application, the terms “first” and “second” etc., are used to distinguish different elements, but do not represent a spatial arrangement or temporal order, etc., of these elements, and these elements should not be limited by these terms. The term “and/or” includes any and all combinations of one or more associated listed terms. The terms “comprise”, “include”, “have”, etc., refer to the presence of described features, elements, components, or assemblies, but do not exclude the presence or addition of one or more other features, elements, components, or assemblies.

In the embodiments of the present application, the singular forms “a” and “the”, etc., include plural forms, and should be broadly construed as “a type of” or “a class of” rather than being limited to the meaning of “one”. Furthermore, the term “the” should be construed as including both the singular and plural forms, unless otherwise specified in the context. In addition, the term “according to” should be construed as “at least in part according to . . . ” and the term “on the basis of” should be construed as “at least in part on the basis of . . . ”, unless otherwise specified in the context.

The features described and/or illustrated for one embodiment may be used in one or more other embodiments in the same or similar manner, be combined with features in other embodiments, or replace features in other embodiments. The term “include/comprise” when used herein refers to the presence of features, integrated components, steps, or assemblies, but does not preclude the presence or addition of one or more other features, integrated components, steps, or assemblies.

The device described herein that obtains medical imaging data may be applicable to various medical imaging modalities, including but not limited to, CT (computed tomography) devices, PET (positron emission tomography)-CT, or any other suitable medical imaging devices.

The system obtaining the medical imaging data may include the aforementioned medical imaging device, and may include a separate computer device connected to the medical imaging device, and may further include a computer device connected to an Internet cloud, the computer device being connected by means of the Internet to the medical imaging device or a memory for storing medical images. The imaging method may be independently or jointly implemented by the aforementioned medical imaging device, the computer device connected to the medical imaging device, and the computer device connected to the Internet cloud.

Exemplarily, the embodiments of the present application are described below in conjunction with an X-ray computed tomography (CT) device. Those skilled in the art would appreciate that the embodiments of the present application can also be applied to other medical imaging devices.

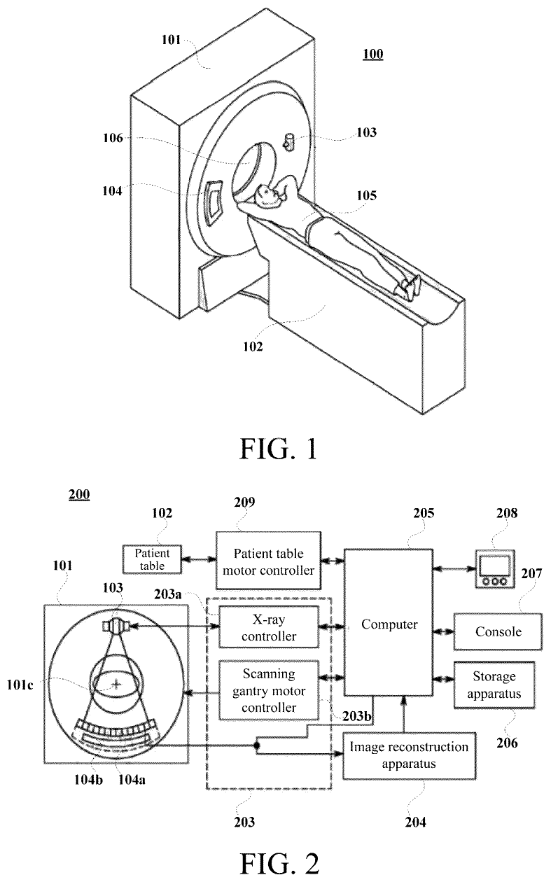

FIG. 1 is a schematic diagram of a CT imaging device according to an embodiment of the present application, and schematically shows a CT imaging device 100. As shown in FIG. 1, the CT imaging device 100 includes a scanning gantry 101 and a patient table 102. The scanning gantry 101 has an X-ray source 103, and the X-ray source 103 projects an X-ray beam toward a collimator or detector assembly 104 on the opposite side of the scanning gantry 101. A test subject 105 can lie flat on the patient table 102 and be moved into a scanning gantry opening 106 along with the patient table 102. Medical image data of the test subject 105 can be acquired by scanning performed by the X-ray source 103.

FIG. 2 is a schematic diagram of a CT imaging system according to an embodiment of the present application, and schematically shows a block diagram of a CT imaging system 200. As shown in FIG. 2, the detector assembly 104 includes a plurality of detector units 104a and a data acquisition system (DAS) 104b. The plurality of detector units 104a sense a projected X-ray passing through the test subject 105.

The DAS 104b, according to the sensing of the detector units 104a, converts collected information into projection data for subsequent processing. During the scanning for acquiring the X-ray projection data, the scanning gantry 101 and components mounted thereon rotate around a rotation center 101c.

The rotation of the scanning gantry 101 and the operation of the X-ray source 103 are controlled by a control mechanism 203 of the CT imaging system 200. The control mechanism 203 includes an X-ray controller 203a that provides power and a timing signal to the X-ray source 103 and a scanning gantry motor controller 203b that controls the rotational speed and position of the scanning gantry 101. An image reconstruction apparatus 204 receives the projection data from the DAS 104b and performs image reconstruction. A reconstructed image is transmitted as an input to a computer 205, and the computer 205 stores the image in a mass storage apparatus 206.

The computer 205 also receives commands and scanning parameters from an operator by means of a console 207. The console 207 has an operator interface of a certain form, such as a keyboard, a mouse, a voice activated controller, or any other suitable input apparatus. An associated display 208 allows the operator to observe the reconstructed image and other data from the computer 205. The commands and parameters provided by the operator are used by the computer 205 to provide control signals and information to the DAS 104b, the X-ray controller 203a, and the scanning gantry motor controller 203b. Additionally, the computer 205 operates a patient table motor controller 209 which controls the patient table 102 so as to position the test subject 105 and the scanning gantry 101. In particular, the patient table 102 moves the test subject 105 as a whole or in part to pass through the scanning gantry opening 106 in FIG. 1.

The device and system for acquiring medical image data (which may also be referred to as medical images or medical image data) according to the embodiments of the present application are schematically described above, but the present application is not limited thereto. The medical imaging device may be a CT device, a PET-CT, or any other suitable imaging device. A storage device may be located within the medical imaging device, in a server outside the medical imaging device, in an independent medical imaging storage system (such as a Picture Archiving and Communication System (PACS)), and/or in a remote cloud storage system.

In addition, a medical imaging workstation may be provided locally to the medical imaging device, that is, the medical imaging workstation is provided close to the medical imaging device, and the two may both be located in a scanning room, an imaging department, or the same hospital. In contrast, a medical image cloud platform analysis system may be positioned distant from the medical imaging device, e.g., arranged at a cloud end that is in communication with the medical imaging device.

As an example, after a medical institution completes an imaging scan using the medical imaging device, data obtained by scanning is stored in a storage device. A medical imaging workstation may directly read the data obtained by scanning and perform image processing by means of a processor thereof. As another example, the medical image cloud platform analysis system may read a medical image in the storage device by means of remote communication to provide “software as a service (SAAS)”. The SAAS may exist between hospitals, between a hospital and an imaging center, or between a hospital and a third-party online diagnosis and treatment service provider.

Medical image scanning is schematically illustrated above, and the embodiments of the present application are described in detail below in view of the accompanying drawings.

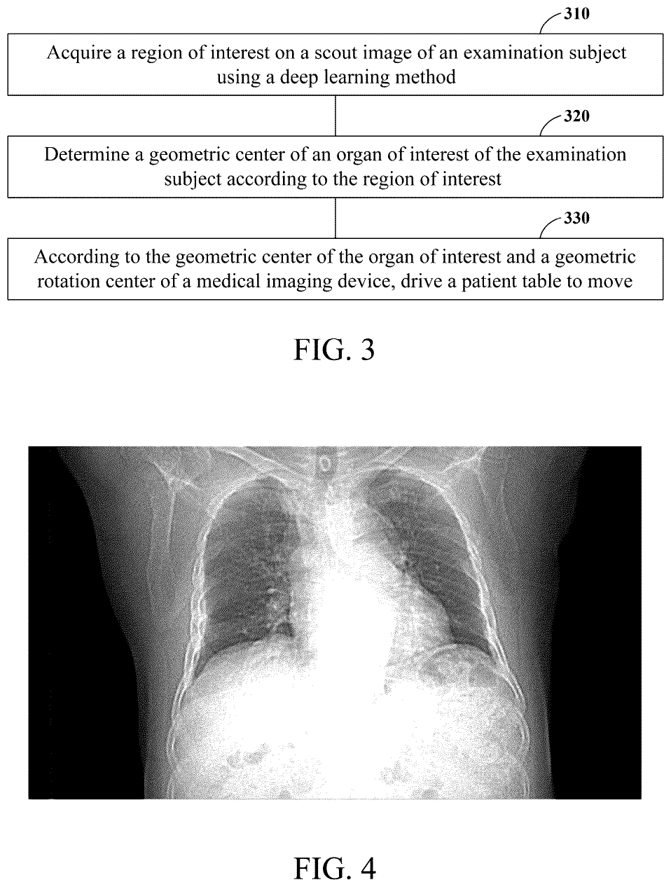

An embodiment of the present application provides a medical imaging method. FIG. 3 is a schematic diagram of a medical imaging method according to an embodiment of the present application. As shown in FIG. 3, the method includes:
310: Acquiring a region of interest on a scout image of an examination subject using a deep learning method;
320: Determining a geometric center of an organ of interest of the examination subject according to the region of interest; and
330: According to the geometric center of the organ of interest and a geometric rotation center of a medical imaging device, driving a patient table to move.

It should be noted that FIG. 3 merely schematically illustrates the embodiments of the present application, but the present application is not limited thereto. For example, some other operations may also be added, or some of the operations may be omitted. Those skilled in the art could make appropriate variations according to the above content, rather than being limited by the above disclosure of FIG. 3.

In the embodiment described above, the geometric center of the organ of interest of the examination subject is determined according to the region of interest on the scout image, and the patient table is driven to move according to the geometric center of the organ of interest and the geometric rotation center of the medical imaging device, thereby achieving automatic movement of the patient table. Therefore, the geometric center of the organ of interest is close to the geometric rotation center of the medical imaging device (i.e., the CT gantry), so that a diagnostic image has higher image quality, including a higher image resolution. In addition, rays passing through the geometric rotation center of the medical imaging device are fully utilized, accordingly improving the dose efficiency of the rays.

In the embodiment of the present application, the scout image of the examination subject is obtained by performing CT scanning on an examination site of the examination subject using the medical imaging device such as a CT device.

In some embodiments, in step 310, the region of interest is acquired from at least two scout images photographed at different angles, that is, two scout images with different imaging angles are used as inputs to a network model used in the deep learning method, so as to separately acquire the region of interest. For example, two scout images whose imaging angles are 0 degrees and 90 degrees, respectively, are used. However, the present application is not limited thereto, and two scout images whose imaging angles are respectively other angles may also be used, or more than two scout images may be used, to respectively obtain the regions of interest.

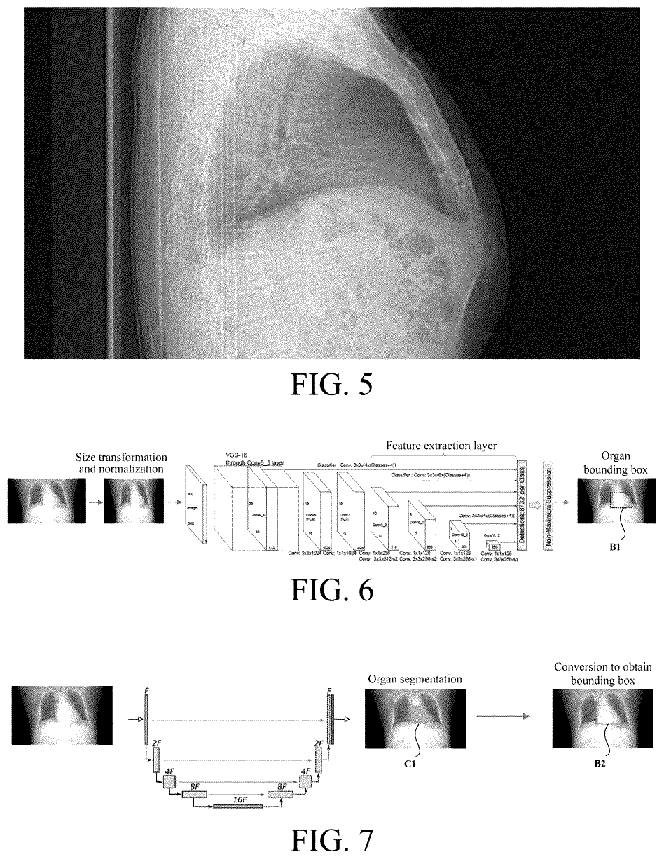

FIG. 4 is a schematic diagram of a scout image of the chest, and in the example of FIG. 4, the imaging angle is 0 degrees. FIG. 5 is another schematic diagram of a scout image of the chest, and in the example of FIG. 5, the imaging angle is 90 degrees. In the examples of FIG. 4 and FIG. 5, the chest of the examination subject is used as the examination site as an example. However, the present application is not limited thereto, and the examination site may be another part of the examination subject, such as the leg or back.

In the embodiments described above, there is no limitation on the network model (i.e., a deep learning network) used in the deep learning method. For example, a convolutional neural network model, a deep belief network model, a stacked autoencoder network model, or the like may be used. Typically, for example, a trained U-net may be used. For details, reference may be made to the related technology, and descriptions are omitted herein.

In some embodiments, before the region of interest on the scout image of the examination subject is obtained using the deep learning method, size transformation and/or normalization processing may be performed on the scout image, so that the processed scout image can be adapted to the architecture and requirements of the network model. However, the present application is not limited thereto, and in some embodiments, the input scout image itself can already meet the requirements of the network model. In this case, there is no need to perform size transformation/normalization processing or the like on the scout image.

In some embodiments, the region of interest obtained by means of the deep learning method is a bounding box for the organ of interest. That is, the bounding box for the organ of interest can be directly extracted by means of the deep learning method.

FIG. 6 is a schematic diagram of a bounding box B1 for an organ of interest, said bounding box being obtained by performing size transformation and normalization on an input scout image, and then performing feature extraction on the scout image by means of a deep learning network.

In some other embodiments, the region of interest obtained by means of the deep learning method is a contour of the organ of interest. That is, the contour of the organ of interest can be obtained by means of the deep learning method. In these embodiments, the contour of the organ of interest may be further converted, to obtain a bounding box for the organ of interest. The specific conversion method is not limited in the present application. For example, the maximum boundary values of top, bottom, left, and right sides of the contour may be taken to draw perpendicular lines to obtain a bounding box corresponding to the contour. For another example, the sum of the maximum boundary values of the top, bottom, left, and right sides of the contour and a predetermined value may be used as a bounding box corresponding to the contour.

FIG. 7 is a schematic diagram of a bounding box B2 for an organ of interest, said bounding box being obtained by performing feature extraction on an input scout image by means of a deep learning network to obtain a contour C1 of the organ of interest, and then converting the contour C1.

In the examples of FIG. 6 and FIG. 7, the 0-degree scout image shown in FIG. 4 is used as an example, but processing similar to that of FIG. 6 or FIG. 7 may also be performed on the 90-degree scout image shown in FIG. 5, so as to obtain a bounding box for the organ of interest.

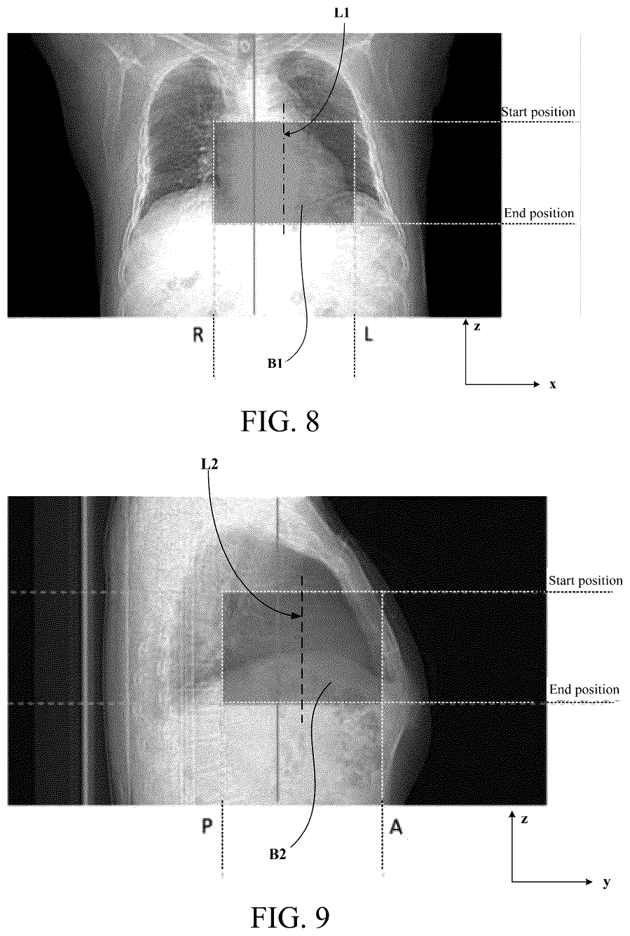

FIG. 8 is a schematic diagram of a bounding box for an organ of interest obtained based on the 0-degree scout image shown in FIG. 4, and FIG. 9 is a schematic diagram of a bounding box for an organ of interest obtained based on the 90-degree scout image shown in FIG. 5.

As shown in FIG. 8, with the use of a deep learning method and a deep learning network, boundaries L and R in an x direction (the left-right movement direction of the patient table) of the bounding box for the organ of interest are obtained, where L represents the left side of the examination subject, and R represents the right side of the examination subject. In the example of FIG. 8, the direction perpendicularly passing through the paper surface is a y direction, that is, the up-down movement direction of the patient table.

As shown in FIG. 9, with the use of a deep learning method and a deep learning network, boundaries A and P in the y direction (the up-down movement direction of the patient table) of the bounding box for the organ of interest are obtained, where A represents the upper side or the front side of the examination subject, and R represents the lower side or the rear side of the examination subject. In the example of FIG. 9, the direction perpendicularly passing through the paper surface is the x direction, that is, the left-right movement direction of the patient table.

In addition, in the examples of FIG. 8 and FIG. 9, a start position and an end position of the organ of interest in a z direction (the front-back movement direction of the patient table) (that is, the direction in which the patient table carrying the examination subject moves in or out of the scanning gantry opening 106) are also marked.

In some embodiments, in step 320, the geometric center of the organ of interest of the examination subject may be determined according to the bounding box for the organ of interest.

For example, a line obtained by intersecting a tangent plane of a center line of a first bounding box obtained from a first scout image of the examination subject with a tangent plane of a center line of a second bounding box obtained from a second scout image of the examination subject may be used as the geometric center of the organ of interest of the examination subject. The tangent plane of the center line of the first bounding box refers to a cross section in a y direction that relates to a center line of the first bounding box relative to an x direction; and the tangent plane of the center line of the second bounding box refers to a cross section in the x direction that relates a center line of the second bounding box relative to the y direction.

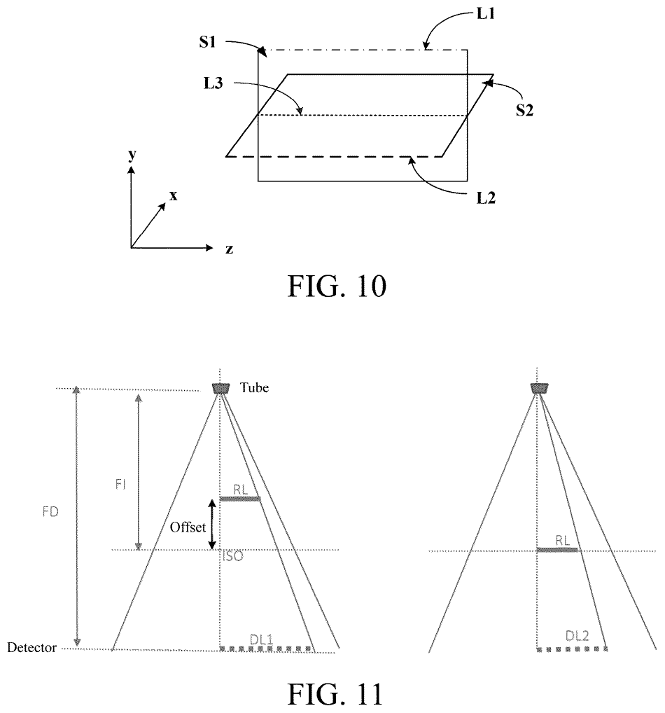

Using the two bounding boxes shown in FIG. 8 and FIG. 9 as an example, FIG. 8 is used as the first scout image, and the bounding box B1 shown in FIG. 8 is the first bounding box, whereas FIG. 9 is used as the second scout image, and the bounding box B2 shown in FIG. 9 is the second bounding box. In this case, the center line of the bounding box B1 refers to a line of the bounding box B1 that equally divides the bounding box B1 in the x direction, and is denoted as L1; similarly, the center line of the bounding box B2 refers to a line of the bounding box B2 that equally divides the bounding box B2 in the y direction, and is denoted as L2. In this case, the tangent plane of the center line L1 of the bounding box B1 refers to a cross section of said center line L1 in the y direction, and is denoted S1, as shown in FIG. 10; similarly, the tangent plane of the center line L2 of the bounding box B2 refers to a cross section of said center line L2 in the x direction, and is denoted as S2, as shown in FIG. 10. In the embodiments of the present application, the geometric center of the organ of interest of the examination subject is a line obtained by intersecting S1 and S2, which is L3 as shown in FIG. 10.

In the embodiments described above, the patient table is driven to move in such a manner that the geometric center of the organ of interest (i.e., the foregoing center line L3) approaches the geometric rotation center of the medical imaging device (e.g., the center of rotation of the CT gantry). In this way, the geometric center of the organ of interest can be brought as close as possible to the geometric rotation center of the medical imaging device, and the image quality and image resolution of a diagnostic image obtained thereby are higher. In addition, rays that pass through the geometric rotation center of the medical imaging device are fully utilized, so that the dose efficiency of the rays is higher.

In the embodiments described above, a reconstruction center and a display field of view of a diagnostic image of the examination subject can be further determined according to the bounding boxes for the organ of interest (for example, the first bounding box and second bounding box).

For example, the origin for coordinates (X, Y) of the reconstruction center of the diagnostic image is the center of the first bounding box in the x direction (the center of R and L) and the center of the second bounding box in y direction (the center of P and A). The origin is expressed by the following formula:

$$( X , Y ) = ( ( R - L ) / 2 , ( P - A ) / 2 ) .$$

For another example, a value of the display field of view (DFOV) of the diagnostic image is the larger among the absolute value of the difference between two boundary positions on the first bounding box in the x direction (abs(R-L)) and the absolute value of the difference between two boundary positions on the second bounding box in the y direction (abs(P-A)). The origin is expressed by the following formula:

$$DFOV = Max ( abs ( R - L ) , abs ( P - A ) ) .$$

In some embodiments, in step 320, the geometric center of the organ of interest of the examination subject may be determined according to the contour of the organ of interest.

For example, a line obtained by intersecting a tangent plane of a center line of a first contour obtained from a first scout image of the examination subject with a tangent plane of a center of a second contour obtained from a second scout image of the examination subject may be used as the geometric center of the organ of interest of the examination subject. The tangent plane of the center line of the first contour refers to a cross section in a y direction that relates to a center line of the first contour relative to an x direction; and the tangent plane of the center line of the second contour refers a cross section in the x direction that relates to a center line of the second contour relative to the y direction.

Using the contour C1 (see the contour C1 shown in FIG. 7) of the organ of interest obtained based on the scout image shown in FIG. 4 and a contour C2 (not shown) of the organ of interest obtained based on the scout image shown in FIG. 5 as an example, FIG. 4 is used as the first scout image, the contour C1 obtained based on FIG. 4 is used as the first contour, FIG. 5 is used as the second scout image, and the contour C2 obtained based on FIG. 5 is used as the second contour.

In the embodiment described above, the center line of the contour C1 refers to a center line between two side boundaries of the contour C1 in the x direction, and the tangent plane of the center line of the contour C1 refers to a cross section of the center line in the y direction; similarly, the center line of the contour C2 refers to a center line between two side boundaries of the contour C2 in the y direction, and the tangent plane of the center line of the contour C2 refers to a cross section of the center line in the x direction.

In the embodiment described above, a reconstruction center and a display field of view of a diagnostic image of the examination subject can be further determined according to the contours of the organ of interest (for example, the first contour and second contour).

For example, the center of the leftmost boundary (most L) and the rightmost boundary (most R) of the contour C1 in the x direction is used as the X coordinate of the reconstruction center, and the center of the foremost boundary (most A) and the rearmost boundary (most P) of the contour C2 in the y direction is used as the Y coordinate of the reconstruction center. The origin is expressed by the following formula:

$$( X , Y ) = ( ( most R - most L ) / 2 , ( most P - most A ) / 2 ) .$$

For another example, the larger of the absolute value of the difference between a leftmost boundary position and a rightmost boundary position on the contour C1 in the x direction (abs (most R-most L)) and the absolute value of the difference between a leftmost boundary position and a rightmost boundary position on the contour C2 in the x direction (abs (most P-most A)) is used as the value of the display field of view DFOV. The origin is expressed by the following formula:

$$DFOV = Max ( abs ( most R - most L ) , abs ( most P - most A ) ) .$$

The foregoing two examples are merely illustrative, and according to different scout images and/or according to different regions of interest obtained based on scout images, the reconstruction center and the display field of view may also be obtained using other methods on the basis of the principles of the foregoing examples. For example, the reconstruction center may be obtained using a method of calculating the center or centroid of a pattern, and then a corresponding display field of view obtained according to the obtained reconstruction center.

In the embodiments of the present application, in some embodiments, the examination subject is not located at or near the geometric rotation center of the medical imaging device, but has an offset from the geometric rotation center of the medical imaging device, which results in an inaccurate display field of the obtained diagnostic image. Therefore, it is necessary to design an adjustment coefficient for the display field of view to adjust the display field of view.

FIG. 11 is a schematic diagram of a display field-of-view adjustment strategy. As shown on the left side of FIG. 11, there is an offset between the position RL of the examination subject and the reconstruction center; accordingly, the obtained display field of view DL1 of the diagnostic image is relatively large. Actually, as shown on the right side of FIG. 11, when the examination subject is located at the reconstruction center, the obtained display field of view DL2) of the diagnostic image is smaller and more accurate than DL1.

In the embodiment described above, an offset value (offset) of a current scout image (as shown on the left side of FIG. 11) may be obtained according to another scout image (as shown on the right side of FIG. 11). Therefore, an adjustment coefficient M for the DFOV may be obtained.

For example, the adjustment coefficient M for the display field of view may be determined according to a distance (F1) from an X-ray tube to the reconstruction center of the diagnostic image and a distance (offset) from the examination subject to the reconstruction center of the diagnostic image.

In the embodiment described above, the adjustment coefficient M for the display field of view may be expressed as the difference between the distance (F1) from the X-ray tube to the reconstruction center of the diagnostic image and the distance (offset) from the examination subject to the reconstruction center of the diagnostic image, divided by the distance (F1) from the X-ray tube to the reconstruction center of the diagnostic image. The origin is expressed by the following formula:

$$M = DL 2 / DL 1 = ( F 1 - Offset ) / F 1$$

In the embodiment described above, an updated value of the display field of view may be determined according to the adjustment coefficient M for the display field of view.

For example, still using the scout images and the bounding boxes B1 and B2 shown in FIG. 8 and FIG. 9 as an example, after the adjustment coefficient M for the display field of view is introduced, the calculation formula of the display field of view is changed to the following:

$$DFOV = Max ( M 1 \times abs ( R - L ) , M 2 \times abs ( P - A ) )$$

Where M1 is an adjustment coefficient for the display field of view corresponding to the scout image shown in FIG. 8, and M2 is a modulation coefficient for the display field of view corresponding to the scout image shown in FIG. 9.

In the embodiment described above, M1 and M2 may be the same or different, that is, adjustment coefficients for the display fields of view of different scout images may be the same or different.

In the embodiments of the present application, an allowable movement range of the patient table varies correspondingly based on differences in the physical statures of examination subjects. In some embodiments, a relationship between the height of the patient table and the allowable movement range may be preset, and then an allowable movement range of the patient table determined according to said relationship and a distance between the geometric center of the organ of interest and the geometric rotation center of the medical imaging device. Further, whether the movement of the patient table exceeds an allowed movement limit is determined, and a control unit drives the patient table to move with reference to the determination result, so that the movement of the patient table does not exceed the allowed movement limit.

In the embodiment described above, the relationship may be represented as a table. For example, the relationship is a tabular configuration file, and the configuration file includes a plurality of patient tables and allowable movement ranges corresponding to the heights of the respective patient tables.

In the embodiments described above, the relationship may also be represented as a fit function of the height of the patient table and the allowable movement range, so that it is possible to determine, according to the height of the patient table, the allowable movement range corresponding to the height of the patient table.

According to the embodiments described above, the relationship between the height of the patient table and the allowable movement range is taken into consideration, thereby avoiding interference between the patient table and a gantry bore during movement, and further improving operability.

The above embodiments merely provide illustrative descriptions of the embodiments of the present application. However, the present application is not limited thereto, and appropriate variations may be made on the basis of the above embodiments. For example, each of the above embodiments may be used independently, or one or more among the above embodiments may be combined.

According to the embodiments of the present application, automatic movement of the patient table is achieved, so that the geometric center of the organ of interest is close to the geometric rotation center of the medical imaging device, enabling diagnostic images to have higher image quality, including a higher image resolution. In addition, rays passing through the geometric rotation center of the medical imaging device are fully utilized, accordingly improving the dose efficiency of the rays.

Embodiments of the present application provide a medical imaging apparatus, and the same content as that of the embodiments of the first aspect is not repeated herein.

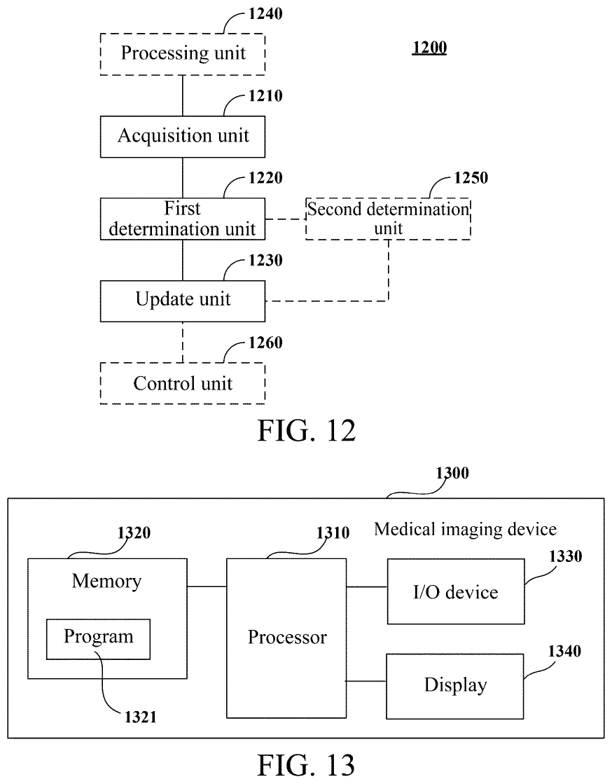

FIG. 12 is a schematic diagram of a medical imaging apparatus according to an embodiment of the present application. As shown in FIG. 12, a medical imaging apparatus 1200 according to an embodiment of the present application includes:

An acquisition unit 1210, which acquires a region of interest on a scout image of an examination subject using a deep learning method;

A first determination unit 1220, which determines a geometric center of an organ of interest of the examination subject according to the region of interest; and

A control unit 1230, which drives a patient table to move according to the geometric center of the organ of interest and a geometric rotation center of a medical imaging device.

In some embodiments, the acquisition unit 1210 acquires the region of interest from at least two scout images photographed at different angles.

In some embodiments, as shown in FIG. 12, the apparatus 1200 further includes:

A processing unit 1240, which performs size transformation and/or normalization processing on the scout image of the examination subject, where the acquisition unit 1210 acquires the region of interest from the scout image processed by the processing unit 1240.

In some embodiments, acquiring the region of interest includes: acquiring a bounding box for the organ of interest of the examination object.

In some embodiments, acquiring the region of interest includes: acquiring a contour of the organ of interest (organ contour).

In the embodiments described above, the acquisition unit 1210 may further convert the contour of the organ of interest, so as to obtain the bounding box for the organ of interest.

In some embodiments, the first determination unit 1220 determines the geometric center of the organ of interest according to the bounding box for the organ of interest. For example, the geometric center of the organ of interest is:

A line obtained by intersecting a tangent plane of a center line of a first bounding box obtained from a first scout image of the examination subject with a tangent plane of a center line of a second bounding box obtained from a second scout image of the examination subject, where the tangent plane of the center line of the first bounding box refers a cross section in a y direction that relates to a center line of the first bounding box relative to an x direction; the tangent plane of the center line of the second bounding box refers to a cross section in the x direction that relates to a center line of the second bounding box relative to the y direction; and the x direction is a left-right movement direction of the patient table, and the y direction is an up-down movement direction of the patient table.

In the embodiments described above, the first determination unit 1220 may further determine a reconstruction center and a display field of view of a diagnostic image of the examination subject according to the bounding box for the organ of interest. For example, the first determination unit 1220 determines the reconstruction center and the display field of view of the diagnostic image of the examination subject according to the first bounding box and the second bounding box.

In the embodiments described above, the center of the first bounding box in the x direction and the center of the second bounding box in the y direction are used as the origin for coordinates of the reconstruction center of the diagnostic image; and the larger among the absolute value of the difference between two side boundary positions on the first bounding box in the x direction, and the absolute value of the difference between two side boundary positions on the second bounding box in the y direction is used as a value of the display field of view.

In some embodiments, the first determination unit 1220 determines the geometric center of the organ of interest of the examination subject according to the contour of the organ of interest. For example, the geometric center of the organ of interest of the examination subject is a line obtained by intersecting a tangent plane of a center line of a first contour obtained from a first scout image of the examination subject with a tangent plane of a center line of a second contour obtained from a second scout image of the examination subject, where the tangent plane of the center line of the first contour refers a cross section in a y direction that relates to a center line of the first contour relative to the x direction; the tangent plane of the center line of the second contour refers a cross section in the x direction that relates to a center line of the second contour relative to the y direction; and the x direction is a left-right movement direction of the patient table, and the y direction is an up-down movement direction of the patient table.

In the embodiments described above, the first determination unit 1220 may further determine the reconstruction center and the display field of view of the diagnostic image of the examination subject according to the contour of the organ of interest, for example, determine the reconstruction center and the display field of view of the diagnostic image of the examination subject according to the first contour and the second contour.

In the embodiments described above, the center of the first contour in the x direction and the center of the second contour in the x direction are used as the origin for coordinates of the reconstruction center of the diagnostic image; and the larger among the absolute value of the difference between two side boundary positions on the first contour in the x direction, and the absolute value of the difference between two side boundary positions on the second contour is used as a value of the display field of view.

In some embodiments, as shown in FIG. 12, the apparatus 1200 further includes an update unit 1250, which determines an adjustment coefficient for the display field of view according to a distance (F1) from an X-ray tube to the reconstruction center of the diagnostic image and a distance (offset) from the examination subject to the reconstruction center of the diagnostic image, and determines an updated value of the display field of view according to the adjustment coefficient for the display field of view.

In the embodiments described above, the adjustment coefficient for the display field of view may be the difference between the distance (F1) from the X-ray tube to the reconstruction center of the diagnostic image and the distance (offset) from the examination subject to the reconstruction center of the diagnostic image, divided by the distance (F1) from the X-ray tube to the reconstruction center of the diagnostic image. In the embodiments described above, the adjustment coefficients (M1 and M2) for different scout images may be the same or different.

In some embodiments, as shown in FIG. 12, the apparatus 1200 further includes a second determination unit 1260, which determines whether the movement of the patient table exceeds an allowable movement limit according to a preset relationship between the height of the patient table and an allowable movement range and a determined distance between the geometric center of the organ of interest and the geometric rotation center of the medical imaging device, where the control unit 1230 drives the patient table to move with reference to a determining result of the second determination unit, so that the movement of the patient table does not exceed the allowable movement limit.

In the embodiments described above, the relationship may be a tabular configuration file, and the configuration file includes a plurality of patient tables and allowable movement ranges corresponding to the heights of the respective patient tables. In the embodiments described above, the relationship may also be a fit function of the height of the patient table and the allowable movement range.

For the sake of simplicity, FIG. 12 only exemplarily illustrates the connection relationship or signal direction between various components or modules, but it should be clear to those skilled in the art that various related technologies such as bus connection can be used. The various components or modules can be implemented by means of hardware such as a processor or a memory, etc. The embodiments of the present application are not limited thereto.

The above embodiments merely provide illustrative descriptions of the embodiments of the present application. However, the present application is not limited thereto, and appropriate variations may be made on the basis of the above embodiments. For example, each of the above embodiments may be used independently, or one or more among the above embodiments may be combined.

According to the embodiments of the present application, automatic movement of the patient table is achieved, so that the geometric center of the organ of interest is close to the geometric rotation center of the medical imaging device, enabling diagnostic images to have higher image quality, including a higher image resolution. In addition, rays passing through the geometric rotation center of the medical imaging device are fully utilized, accordingly improving the dose efficiency of the rays.

Embodiments of the present application provide a medical imaging device, including the medical imaging apparatus 1200 as described in the embodiments of the second aspect, the content of which is incorporated herein and will not be described again. The medical imaging device may, for example, be a computer, a server, a workstation, a laptop computer, a smart phone, or the like. However, the embodiments of the present application are not limited thereto.

FIG. 13 is a schematic diagram of a medical imaging device according to an embodiment of the present application. As shown in FIG. 13, a medical imaging device 1300 may include one or more processors (for example, central processing units (CPU)) 1310 and one or more memories 1320. The memory 1320 is coupled to the processor 1310. The memory 1320 may store various types of data. In addition, the memory further stores a program 1321 for information processing, and executes the program 1321 under the control of the processor 1310.

In some embodiments, the functions of the medical imaging apparatus 1200 are integrated into the processor 1310 for implementation. The processor 1310 is configured to implement the medical imaging method according to the embodiments of the first aspect.

In some embodiments, the medical imaging apparatus 1200 and the processor 1310 are configured separately. For example, the medical imaging apparatus 1200 may be configured as a chip connected to the processor 1310, and the functions of the medical imaging apparatus 1200 implemented under the control of the processor 1310.

For example, the processor 1310 is configured to perform the following control: acquiring a region of interest on a scout image of an examination subject using a deep learning method; determining a geometric center of an organ of interest of the examination subject according to the region of interest; and according to the geometric center of the organ of interest and a geometric rotation center of a medical imaging device, driving a patient table to move.

In addition, as shown in FIG. 13, the medical imaging device 1300 may further include: an input/output (I/O) device 1330, a display 1340, and the like. The functions of said components are similar to those in the prior art, and are not described again here. It should be noted that the medical imaging device 1300 does not necessarily include all of the components shown in FIG. 13. In addition, the medical imaging device 1300 may further include components not shown in FIG. 13, for which reference may be made to related technology.

An embodiment of the present application further provides a computer-readable program, where when the program is executed in an electronic device, the program enables a computer to execute, in the electronic device, the medical imaging method as described in the embodiments of the first aspect.

An embodiment of the present application further provides a storage medium storing a computer-readable program, where the computer-readable program enables a computer to execute, in an electronic device, the medical imaging method as described in the embodiments of the first aspect.

The above apparatus and method of the present application can be implemented by hardware, or can be implemented by hardware in combination with software. The present application relates to the foregoing type of computer-readable program. When executed by a logic component, the program causes the logic component to implement the foregoing apparatus or a constituent component, or causes the logic component to implement various methods or steps as described above. The present application further relates to a storage medium for storing the above program, such as a hard disk, a disk, an optical disk, a DVD, a flash memory, etc.

The method/apparatus described in view of the embodiments of the present application may be directly embodied as hardware, a software module executed by a processor, or a combination of the two. For example, one or more of the functional block diagrams and/or one or more combinations of the functional block diagrams shown in the drawings may correspond to either respective software modules or respective hardware modules of a computer program flow. The foregoing software modules may respectively correspond to the steps shown in the figures. The foregoing hardware modules can be implemented, for example, by firming the software modules using a field-programmable gate array (FPGA).

The software modules may be located in a RAM, a flash memory, a ROM, an EPROM, an EEPROM, a register, a hard disk, a portable storage disk, a CD-ROM, or any other form of storage medium known in the art. The storage medium may be coupled to a processor, so that the processor can read information from the storage medium and can write information into the storage medium. Alternatively, the storage medium may be a constituent component of the processor. The processor and the storage medium may be located in an ASIC. The software module may be stored in a memory of a mobile terminal, and may also be stored in a memory card that can be inserted into a mobile terminal. For example, if a device (such as a mobile terminal) uses a large-capacity MEGA-SIM card or a large-capacity flash memory device, the software modules can be stored in the MEGA-SIM card or the large-capacity flash memory apparatus.

One or more of the functional blocks and/or one or more combinations of the functional blocks shown in the accompanying drawings may be implemented as a general-purpose processor, a digital signal processor (DSP), an application-specific integrated circuit (ASIC), a field-programmable gate array (FPGA) or other programmable logic devices, discrete gate or transistor logic devices, a discrete hardware assembly, or any appropriate combination thereof for implementing the functions described in the present application. The one or more functional blocks and/or the one or more combinations of the functional blocks shown in the accompanying drawings may also be implemented as a combination of computing devices, such as a combination of a DSP and a microprocessor, multiple microprocessors, one or more microprocessors in communication combination with a DSP, or any other such configuration.

The present application is described above with reference to specific embodiments. However, it should be clear to those skilled in the art that the foregoing description is merely illustrative and is not intended to limit the scope of protection of the present application. Various variations and modifications may be made by those skilled in the art according to the principle of the present application, and said variations and modifications also fall within the scope of the present application.

1. A medical imaging apparatus, characterized in that the apparatus comprises:
an acquisition unit, which acquires a region of interest on a scout image of an examination subject using a deep learning method;
a first determination unit, which determines a geometric center of an organ of interest of the examination subject according to the region of interest; and
a control unit, which drives a patient table to move according to the geometric center of the organ of interest and a geometric rotation center of a medical imaging device.

2. The medical imaging apparatus according to claim 1, wherein the acquisition unit acquires the region of interest from at least two scout images photographed at different angles.

3. The medical imaging apparatus according to claim 1, further comprising a processing unit, which performs size transformation and/or normalization processing on the scout image of the examination subject, wherein the acquisition unit acquires the region of interest from the scout image processed by the processing unit.

4. The medical imaging apparatus according to claim 1, wherein acquiring the region of interest comprises: acquiring a bounding box for the organ of interest of the examination subject.

5. The medical imaging apparatus according to claim 1, wherein acquiring the region of interest comprises: acquiring a contour of the organ of interest of the examination subject.

6. The medical imaging apparatus according to claim 5, wherein the acquisition unit further converts the contour of the organ of interest, to obtain a bounding box for the organ of interest.

7. The medical imaging apparatus according to claim 4, wherein the first determination unit determines the geometric center of the organ of interest of the examination subject according to the bounding box for the organ of interest, wherein the geometric center of the organ of interest of the examination subject is a line obtained by intersecting a tangent plane of a center line of a first bounding box obtained from a first scout image of the examination subject with a tangent plane of a center line of a second bounding box obtained from a second scout image of the examination subject, wherein the tangent plane of the center line of the first bounding box refers to a cross section in a y direction that relates to a center line of the first bounding box relative to an x direction, and the tangent plane of the center line of the second bounding box refers to a cross section in the x direction that relates to a center line of the second bounding box relative to the y direction, and the x direction is a left-right movement direction of the patient table, and the y direction is an up-down movement direction of the patient table.

8. The medical imaging apparatus according to claim 7, wherein the first determination unit further determines a reconstruction center and a display field of view of a diagnostic image of the examination subject according to the first bounding box and the second bounding box, wherein an origin for coordinates of the reconstruction center of the diagnostic image is a center of the first bounding box in the x direction and a center of the second bounding box in the y direction, and a value of the display field of view of the diagnostic image is a larger among an absolute value of a difference between two side boundary positions on the first bounding box in the x direction, and the absolute value of the difference between two side boundary positions on the second bounding box in the y direction.

9. The medical imaging apparatus according to claim 5, wherein the first determination unit determines the geometric center of the organ of interest of the examination subject according to the contour of the organ of interest, wherein the geometric center of the organ of interest of the examination subject is a line obtained by intersecting a tangent plane of a center line of a first contour obtained from a first scout image of the examination subject with a tangent plane of a center line of a second contour obtained from a second scout image of the examination subject, wherein the tangent plane of the center line of the first contour refers to a cross section in a y direction that relates to a center line of the first contour relative to an x direction, the tangent plane of the center line of the second contour refers to a cross section in the x direction that relates to a center line of the second contour relative to the y direction, and the x direction is a left-right movement direction of the patient table, and the y direction is an up-down movement direction of the patient table.

10. The medical imaging apparatus according to claim 9, wherein the first determination unit further determines a reconstruction center and a display field of view of a diagnostic image of the examination subject according to the first contour and the second contour, wherein an origin for coordinates of the reconstruction center is: a center of the first contour in the x direction and a center of the second contour in the y direction, and a value of the display field of view of the diagnostic image is a larger among an absolute value of a difference between two side boundary positions on the first contour in the x direction and the absolute value of the difference between two side boundary positions on the second contour in the y direction.

11. The medical imaging apparatus according to claim 8, further comprising an update unit, which determines an adjustment coefficient for the display field of view according to a distance from an X-ray tube to the reconstruction center of the diagnostic image, and a distance from the examination subject to the reconstruction center of the diagnostic image, and determines an updated value of the display field of view according to the adjustment coefficient for the display field of view.

12. The medical imaging apparatus according to claim 11, wherein the adjustment coefficient for the display field of view is the difference between the distance from the X-ray tube to the reconstruction center of the diagnostic image and the distance from the examination subject to the reconstruction center of the diagnostic image, divided by the distance between the X-ray tube and the reconstruction center of the diagnostic image.

13. The medical imaging apparatus according to claim 11, wherein adjustment coefficients for different scout images may be the same or different.

14. The medical imaging apparatus according to claim 1, wherein the apparatus further comprises a second determination unit, which determines, according to a preset relationship between a height of the patient table and an allowable movement range and a determined distance between the geometric center of the organ of interest and the geometric rotation center of the medical imaging device, whether movement of the patient table exceeds an allowable movement limit, wherein the control unit drives the patient table to move with reference to a determination result of the second determination unit, so that the movement of the patient table does not exceed the allowable movement limit.

15. The medical imaging apparatus according to claim 14, wherein the relationship is a tabular configuration file, and the configuration file comprises a plurality of patient tables and allowable movement ranges corresponding to the heights of the respective patient tables.

16. The medical imaging apparatus according to claim 14, wherein the relationship is a fit function of the height of the patient table and the allowable movement range.

17. A medical imaging method, characterized in that the method comprises:
obtaining a region of interest on a scout image of an examination subject using a deep learning method, determining a geometric center of an organ of interest of the examination subject according to the region of interest, and according to the geometric center of the organ of interest and a geometric rotation center of a medical imaging device, driving a patient table to move.

18. The method according to claim 17, further comprising determining a reconstruction center and a display field of view of a diagnostic image of the examination subject according to the organ of interest.
